(19) United States
(12) Patent Application Publication
(10) Pub. No.: US 2022/0306626 A1
(43) Pub. Date: Sep. 29, 2022

(54) CRYSTALLINE FORM OF A DIHYDROCHLORIDE SALT OF A JAK INHIBITOR COMPOUND

(71) Applicant: Theravance Biopharma R&D IP, LLC, South San Francisco, CA (US)

(72) Inventors: Jerry Nzerem, South San Francisco, CA (US); Marta Dabros, Foster City, CA (US); Venkat R. Thalladi, Foster City, CA (US); Michael Simeone, San Francisco, CA (US)

(21) Appl. No.: 17/704,502

(22) Filed: Mar. 25, 2022

Related U.S. Application Data
(60) Provisional application No. 63/166,800, filed on Mar. 26, 2021.

(51) Int. Cl. C07D 471/04

(52) U.S. Cl. CPC C07D 471/04; C07B 2200/13

(57) ABSTRACT

Provided herein is a crystalline form of the dihydrochloride salt of 5-ethyl-2-fluoro-4-(3-(5-(1-methylpiperidin-4-yl)-4,5,6,7-tetrahydro-1H-imidazo[4,5-c]pyridin-2-yl)-1H-indazol-6-yl)phenol. Also provided herein are pharmaceutical compositions comprising such crystalline form, methods of using such crystalline form to treat respiratory diseases, and processes useful for preparing such crystalline form.

### CROSS REFERENCE TO RELATED APPLICATIONS

This application claims the benefit under 35 U.S.C. § 119(e) of U.S. Provisional Application No. 63/166,800, filed Mar. 26, 2021, which is incorporated herein by reference in its entirety.

### FIELD

Provided herein is a crystalline form of a dihydrochloride salt of a JAK inhibitor compound useful for treating respiratory and other diseases. Also provided herein are pharmaceutical compositions comprising such compound, methods of using the salt form to treat, respiratory diseases, and processes useful for preparing such crystalline salt form.

### BACKGROUND

Cytokines are intercellular signaling molecules which include chemokines, interferons, interleukins, lymphokines, and tumor necrosis factor. Cytokines are critical for normal cell growth and immunoregulation but also drive immune-mediated diseases and contribute to the growth of malignant cells. Elevated levels of many cytokines have been implicated in the pathology of a large number of disease or conditions, particularly those diseases characterized by inflammation. Many of the cytokines implicated in disease act through signaling pathways dependent upon the Janus family of tyrosine kinases (JAKs), which signal through the Signal Transducer and Activator of Transcription (STAT) family of transcription factors.

The JAK family comprises four members, JAK1, JAK2, JAK3, and tyrosine kinase 2 (TYK2). Binding of cytokine to a JAK-dependent cytokine receptor induces receptor dimerization which results in phosphorylation of tyrosine residues on the JAK kinase, effecting JAK activation. Phosphorylated JAKs, in turn, bind and phosphorylate various STAT proteins which dimerize, internalize in the cell nucleus and directly modulate gene transcription, leading, among other effects, to the downstream effects associated with inflammatory disease. The JAKs usually associate with cytokine receptors in pairs as homodimers or heterodimers. Specific cytokines are associated with specific JAK pairings. Each of the four members of the JAK family is implicated in the signaling of at least one of the cytokines associated with inflammation.

Asthma is a chronic disease of the airways for which there are no preventions or cures. The disease is characterized by inflammation, fibrosis, hyperresponsiveness, and remodeling of the airways, all of which contribute to airflow limitation. An estimated 300 million people worldwide suffer from asthma and it is estimated that the number of people with asthma will grow by more than 100 million by 2025. Although most patients can achieve control of asthma symptoms with the use of inhaled corticosteroids that may be combined with a leukotriene modifier and/or a long acting beta agonist, there remains a subset of patients with severe asthma whose disease is not controlled by conventional therapies. Cytokines implicated in asthma inflammation which signal through the JAK-STAT pathway include IL-2, IL-3, IL-4, IL-5, IL-6, IL-9, IL-11, IL-13, IL-23, IL-31, IL-27, thymic stromal lymphopoietin (TSLP), interferon-γ (IFNγ) and granulocyte-macrophage colony-stimulating factor (GM-CSF). Inflammation of the airways is characteristic of other respiratory diseases in addition to asthma. Chronic obstructive pulmonary disease (COPD), cystic fibrosis (CF), pneumonitis, interstitial lung diseases (including idiopathic pulmonary fibrosis), acute lung injury, acute respiratory distress syndrome, bronchitis, emphysema, and bronchiolitis obliterans are also respiratory tract diseases in which the pathophysiology is believed to be related to JAK-signaling cytokines.

Commonly assigned U.S. application Ser. No. 15/341,226, filed on Nov. 2, 2016, published as US 2017/0121327, discloses diamino compounds useful as JAK inhibitors. In particular, the compound 5-ethyl-2-fluoro-4-(3-(5-(1-methylpiperidin-4-yl)-4,5,6,7-tetrahydro-1H-imidazo[4,5-c]pyridin-2-yl)-1H-indazol-6-yl)phenol (compound 1)

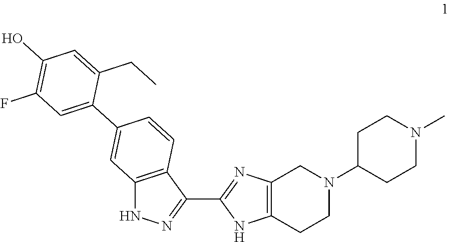 [SMILES: CCc1cc(O)c(F)cc1-c1ccc2c(-c3nc4c([nH]3)CCN(C3CCN(C)CC3)C4)n[nH]c2c1]

is specifically disclosed in the application as a potent pan-JAK inhibitor.

To effectively use this compound as a therapeutic agent, it would be desirable to have a crystalline solid-state salt form. For example, it would be highly desirable to have a physical form that is thermally stable at reasonably high temperature, thereby facilitating processing and storage of the material. Crystalline solids are generally preferred over amorphous forms, for enhancing purity and stability of the manufactured product. However, the formation of crystalline forms of organic compounds is highly unpredictable. No reliable methods exist for predicting which, if any, form of an organic compound will be crystalline. Moreover, no methods exist for predicting which, if any, crystalline form will have the physically properties desired for use as pharmaceutical agents. A need exists for stable crystalline salt forms of compound 1.

### SUMMARY

Provided herein is a crystalline form of the dihydrochloride salt of 5-ethyl-2-fluoro-4-(3-(5-(1-methylpiperidin-4-yl)-4,5,6,7-tetrahydro-1H-imidazo[4,5-c]pyridin-2-yl)-1H-indazol-6-yl)phenol (1).

The crystalline form has been found to have a melting temperature with an onset at 312.6° C., and a peak endotherm at 325.7° C.

The crystalline form exhibits a total moisture uptake of about 0.8% when exposed to a range of relative humidity between about 5% and about 90% at room temperature.

Among other uses, the crystalline solid form of the disclosure is expected to be useful for preparing pharmaceutical compositions for treating or ameliorating diseases amenable to treatment with a JAK inhibitor, in particular respiratory diseases. Accordingly, provided herein is a pharmaceutical composition comprising a pharmaceutically-acceptable carrier and the crystalline form of the dihydrochloride salt of 5-ethyl-2-fluoro-4-(3-(5-(1-methylpiperidin-4-yl)-4,5,6,7-tetrahydro-1H-imidazo[4,5-c]pyridin-2-yl)-1H-indazol-6-yl)phenol.

Also provided herein is a method of treating respiratory diseases, in particular, asthma, in a mammal, the method comprising administering to the mammal a crystalline solid form or a pharmaceutical composition of the disclosure. In separate and distinct aspects, the disclosure also provides synthetic processes useful for preparing the crystalline form of the disclosure.

Also provided is a crystalline solid form of the present disclosure as described herein for use in medical therapy, as well as the use of a crystalline solid form of the present disclosure in the manufacture of a formulation or medicament for treating respiratory diseases in a mammal

### BRIEF DESCRIPTION OF THE DRAWINGS

Various aspects of the present disclosure are illustrated by reference to the accompanying drawings.

FIG. 1 shows a powder x-ray diffraction (PXRD) pattern of the crystalline form of the dihydrochloride salt of 5-ethyl-2-fluoro-4-(3-(5-(1-methylpiperidin-4-yl)-4,5,6,7-tetrahydro-1H-imidazo[4,5-c]pyridin-2-yl)-1H-indazol-6-yl)phenol.

FIG. 2 shows a differential scanning calorimetry (DSC) thermogram of the crystalline form of the dihydrochloride salt of 5-ethyl-2-fluoro-4-(3-(5-(1-methylpiperidin-4-yl)-4,5,6,7-tetrahydro-1H-imidazo[4,5-c]pyridin-2-yl)-1H-indazol-6-yl)phenol.

FIG. 3 shows a thermal gravimetric analysis (TGA) plot of the crystalline form of the dihydrochloride salt of 5-ethyl-2-fluoro-4-(3-(5-(1-methylpiperidin-4-yl)-4,5,6,7-tetrahydro-1H-imidazo[4,5-c]pyridin-2-yl)-1H-indazol-6-yl)phenol.

FIG. 4 shows a dynamic moisture sorption (DMS) isotherm of the crystalline form of the dihydrochloride salt of 5-ethyl-2-fluoro-4-(3-(5-(1-methylpiperidin-4-yl)-4,5,6,7-tetrahydro-1H-imidazo[4,5-c]pyridin-2-yl)-1H-indazol-6-yl)phenol observed at a temperature of about 25° C.

### DETAILED DESCRIPTION

### Definitions

In this disclosure, including its various aspects and embodiments, the following terms have the following meanings, unless otherwise indicated.

The term “therapeutically effective amount” means an amount sufficient to effect treatment when administered to a patient in need of treatment.

The term “treating” or “treatment” means ameliorating or suppressing the medical condition, disease or disorder being treated (e.g., a respiratory disease) in a patient (particularly a human); or alleviating the symptoms of the medical condition, disease or disorder.

The term “about,” in some embodiments, means±5 percent of the specified value. In some embodiments, the term “about” means±2 percent of the specified value. In some embodiments, the term “about” means±1 percent of the specified value. In some embodiments, the term “about” means±10 percent of the specified value.

It must be noted that, as used in the specification and appended claims, the singular forms “a”, “an”, “one”, and “the” may include plural references, unless the content clearly dictates otherwise.

Naming Convention

Compound 1 is designated as 5-ethyl-2-fluoro-4-(3-(5-(1-methylpiperidin-4-yl)-4,5,6,7-tetrahydro-1H-imidazo[4,5-c]pyridin-2-yl)-1H-indazol-6-yl)phenol according to IUPAC conventions as implemented in ChemDraw software (PerkinElmer, Inc., Cambridge, Mass.).

Furthermore, the imidazo portion of the tetrahydroimidazopyridine moiety in the structure of compound 1 exists in tautomeric forms, illustrated below for a fragment of the compound of Example 1

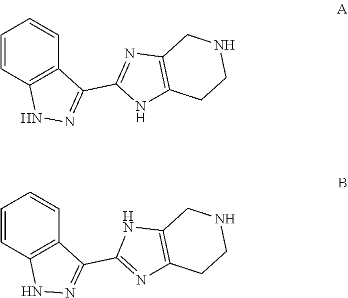 [SMILES: c1ccc2c(-c3nc4c([nH]3)CCNC4)n[nH]c2c1 ; c1ccc2c(-c3nc4c([nH]3)CNCC4)n[nH]c2c1]

According to the IUPAC convention, these representations give rise to different numbering of the atoms of the imidazole portion: 2-(1H-indazol-3-yl)-4,5,6,7-tetrahydro-1H-imidazo[4,5-c]pyridine (structure A) vs. 2-(1H-indazol-3-yl)-4,5,6,7-tetrahydro-3H-imidazo[4,5-c]pyridine (structure B). It will be understood that although structures are shown, or named, in a particular form, the disclosure also includes the tautomer thereof.

Crystalline Form

Provided herein is a crystalline form of the dihydrochloride salt of the compound of formula 1:

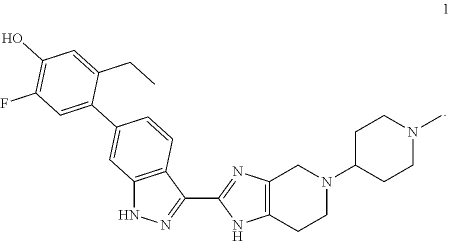 [SMILES: CCc1cc(O)c(F)cc1-c1ccc2c(-c3nc4c([nH]3)CCN(C3CCN(C)CC3)C4)n[nH]c2c1]

In one embodiment, the crystalline dihydrochloride form is characterized by a powder X-ray diffraction pattern comprising diffraction peaks at 2θ values of 5.99±0.2, 11.98±0.2, 17.67±0.2, and 18.02±0.2. In some embodiments, the powder X-ray diffraction pattern is further characterized by having one additional diffraction peaks at a 2θ value of 22.17±0.2. In some embodiments, the powder X-ray diffraction pattern is further characterized by having two additional diffraction peaks at 2θ values of 12.47±0.2, and 21.22±0.2. In some embodiments, the powder X-ray diffraction pattern is further characterized by having two additional diffraction peaks at a 2θ values of 7.74±0.2, and 8.48±0.2. In some embodiments, the powder X-ray diffraction pattern is further characterized by having two or more additional diffraction peaks at 2θ values selected from 7.74±0.2, 8.48±0.2, 12.47±0.2, 21.22±0.2, and 22.17±0.2. In some embodiments, the powder X-ray diffraction pattern is further characterized by having two or more additional diffraction peaks at 2θ values selected from 13.11±0.2, 15.12±0.2, 15.53±0.2, 19.45±0.2, 19.77±0.2, 20.64±0.2, 21.48±0.2, 23.66±0.2, 24.99±0.2, 26.84±0.2, 27.29±0.2, and 28.13±0.2.

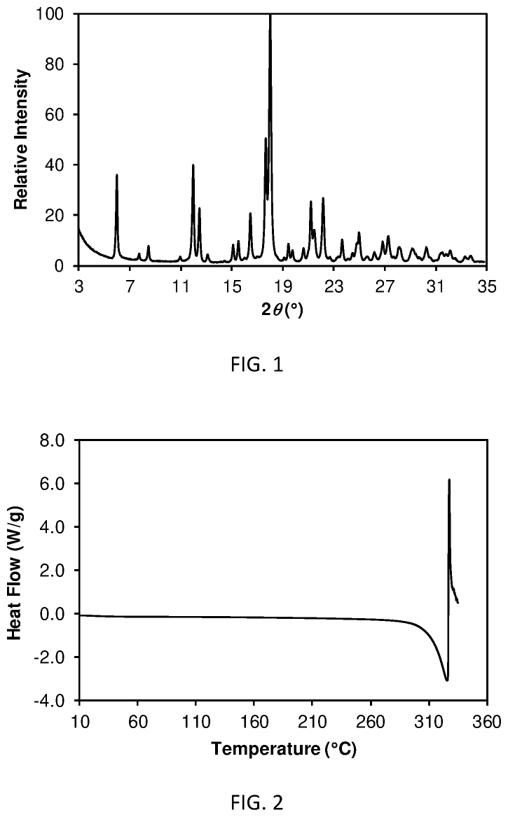

As is well known in the field of powder X-ray diffraction, peak positions of PXRD spectra are relatively less sensitive to experimental details, such as details of sample preparation and instrument geometry, than are the relative peak heights. Accordingly, in some embodiments, the crystalline form is characterized by a powder X-ray diffraction pattern in which the peak positions are substantially in accordance with the peak positions of the pattern shown in FIG. 1. In another aspect, the crystalline form is characterized by its behavior when exposed to high temperature. In some embodiments, the crystalline form is characterized by a differential scanning calorimetry trace recorded at a heating rate of 10° C. per minute which shows a melting temperature with an onset at 312.6° C., and a peak endotherm (maximum in endothermic heat flow) at 325.7° C. In some embodiments, the crystalline form is characterized by a differential scanning calorimetry trace substantially in accordance with that shown in FIG. 2.

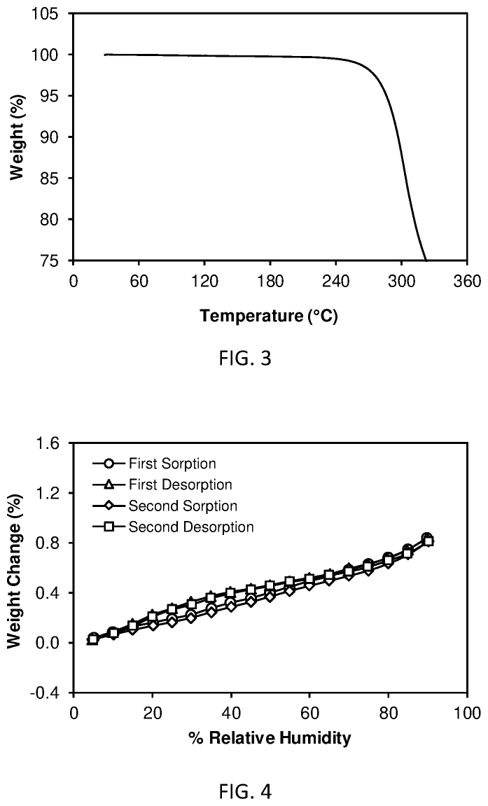

The thermal gravimetric analysis (TGA) trace of FIG. 3 shows decomposition at an onset temperature of about 285° C.

The present crystalline form has been demonstrated to have a reversible sorption/desorption profile with a slight propensity for hygroscopicity. The crystalline form exhibited total moisture uptake of about 0.8% when exposed to a range of relative humidity between about 5% and about 90% at room temperature as shown in FIG. 4. There was no change in form after two cycles of moisture sorption and desorption.

The present crystalline form exhibited good solubility in water and simulated lung fluid.

In some embodiments, provided herein is a pharmaceutical composition comprising the compound of Formula I, wherein at least 95% of the compound of Formula I is the crystalline dihydrochloride form described above. In some embodiments, provided herein is a pharmaceutical composition comprising the compound of Formula I, wherein at least 96% of the compound of Formula I is the crystalline dihydrochloride form described above. In some embodiments, provided herein is a pharmaceutical composition comprising the compound of Formula I, wherein at least 97% of the compound of Formula I is the crystalline dihydrochloride form described above. In some embodiments, provided herein is a pharmaceutical composition comprising the compound of Formula I, wherein at least 98% of the compound of Formula I is the crystalline dihydrochloride form described above. In some embodiments, provided herein is a pharmaceutical composition comprising the compound of Formula I, wherein at least 99% of the compound of Formula I is the crystalline dihydrochloride form described above.

Synthetic Procedures

The crystalline form is conveniently prepared by heating 5-ethyl-2-fluoro-4-(3-(4,5,6,7-tetrahydro-1H-imidazo[4,5-c]pyridin-2-yl)-1H-indazol-6-yl)phenol crystalline hydrate (preparation of the crystalline hydrate is shown in U.S. application Ser. No. 15/341,226 filed on Nov. 2, 2016, published as US 2017/0121327) in DMSO and ethanol (at a 1:2 to 3 ratio of DMSO:ethanol, for example at about a 1:2.4 ratio) to about 55° C., followed by cooling off to about 25° C. and addition of hydrochloric acid (2 to 2.5 equivalents, for example 2.1 equivalents) and ethanol at about 25° C. The solid obtained is then filtered, washed with ethanol and dried. Ethanol and water (at a ratio of 15 to 25:1, for example about 19:1) are added to the solid and the mixture is stirred at about 40° C. for 12-36 hours (for example about 24 h). The resulting suspension is then filtered and washed with ethanol to provide the dihydrochloride salt form.

Accordingly, provided herein is a method of preparing the crystalline form comprising:

(a) mixing 5-ethyl-2-fluoro-4-(3-(4,5,6,7-tetrahydro-1H-imidazo[4,5-c]pyridin-2-yl)-1H-indazol-6-yl)phenol, or a hydrate or solvate thereof, in DMSO and ethanol, with optional heating, to give a mixture,

(b) to the mixture obtained in step (a), adding 2 equivalents or an excess of hydrochloric acid in ethanol to produce a suspension,

(c) isolating the solid from the suspension of step (b),

(d) adding ethanol and water to the solid of step (c) and stirring the mixture obtained to give a suspension, and

(e) isolating the crystalline form from the suspension of step (d).

Also provided herein is a method of preparing the crystalline form comprising:

(a) heating at 55° C.±10° C. 5-ethyl-2-fluoro-4-(3-(4,5,6,7-tetrahydro-1H-imidazo[4,5-c]pyridin-2-yl)-1H-indazol-6-yl)phenol, or a hydrate or solvate thereof, in DMSO and ethanol, wherein the ratio of DMSO:ethanol is 1:2 to 3, to give a mixture,

(b) cooling off the mixture obtained in step (a) to about 25° C. and adding 2 to 2.5 equivalents of hydrochloric acid in ethanol at 25° C.±10° C. to produce a suspension,

(c) filtering the suspension of step (b) to give a solid,

(d) adding ethanol and water at a ratio of ethanol:water of 15 to 25:1 to the solid of step (c) and stirring the mixture obtained at 25° C.±10° C. for 12-36 hours to give a suspension, and

(e) isolating the crystalline form from the suspension of step (d).

In some embodiments, the method involves drying the solid of step (c) at 50° C.±10° C. before performing step (d). In some embodiments, the 5-ethyl-2-fluoro-4-(3-(4,5,6,7-tetrahydro-1H-imidazo[4,5-c]pyridin-2-yl)-1H-indazol-6-yl)phenol used in step (a) is the hydrate.

Pharmaceutical Compositions

The crystalline dihydrochloride salt form of the present disclosure is typically used in the form of a pharmaceutical composition or formulation. Such pharmaceutical compositions may advantageously be administered to a patient by inhalation. In addition, pharmaceutical compositions may be administered by any acceptable route of administration including, but not limited to, oral, topical (including transdermal), rectal, nasal, and parenteral modes of administration.

Accordingly, provided herein is a pharmaceutical composition comprising a pharmaceutically-acceptable carrier or excipient and a crystalline form of the dihydrochloride salt of compound 1. Optionally, such pharmaceutical compositions may contain other therapeutic and/or formulating agents if desired. When discussing compositions and uses thereof, the crystalline solid form of the present disclosure may also be referred to herein as the “active agent”.

The pharmaceutical compositions of the present disclosure typically contain a therapeutically effective amount of the crystalline form of the present disclosure. Those skilled in the art will recognize, however, that a pharmaceutical composition may contain more than a therapeutically effective amount, i.e., bulk compositions, or less than a therapeutically effective amount, i.e., individual unit doses designed for multiple administration to achieve a therapeutically effective amount.

Typically, such pharmaceutical compositions will contain from about 0.01 to about 95% by weight of the active agent; including, for example, from about 0.05 to about 30% by weight; and from about 0.1% to about 10% by weight of the active agent.

Any conventional carrier or excipient may be used in the pharmaceutical compositions of the present disclosure. The choice of a particular carrier or excipient, or combinations of carriers or excipients, will depend on the mode of administration being used to treat a particular patient or type of medical condition or disease state. In this regard, the preparation of a suitable pharmaceutical composition for a particular mode of administration is well within the scope of those skilled in the pharmaceutical arts. Additionally, the carriers or excipients used in the pharmaceutical compositions of this disclosure are commercially-available. By way of further illustration, conventional formulation techniques are described in Remington: The Science and Practice of Pharmacy, 20th Edition, Lippincott Williams & White, Baltimore, Md. (2000); and H. C. Ansel et al., Pharmaceutical Dosage Forms and Drug Delivery Systems, 7th Edition, Lippincott Williams & White, Baltimore, Md. (1999).

Representative examples of materials which can serve as pharmaceutically acceptable carriers include, but are not limited to, the following: sugars, such as lactose, glucose and sucrose; starches, such as corn starch and potato starch; cellulose, such as microcrystalline cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical compositions.

Pharmaceutical compositions are typically prepared by thoroughly and intimately mixing or blending the active agent with a pharmaceutically-acceptable carrier and one or more optional ingredients. The resulting uniformly blended mixture can then be shaped or loaded into tablets, capsules, pills and the like using conventional procedures and equipment.

In one aspect, the pharmaceutical composition is suitable for inhaled administration. Pharmaceutical compositions for inhaled administration are typically in the form of an aerosol or a powder. Such compositions are generally administered using inhaler delivery devices, such as a dry powder inhaler (DPI), a metered-dose inhaler (MDI), a nebulizer inhaler, or a similar delivery device.

In a particular embodiment, the pharmaceutical composition is administered by inhalation using a dry powder inhaler. Such dry powder inhalers typically administer the pharmaceutical composition as a free-flowing powder that is dispersed in a patient's air-stream during inspiration. In order to achieve a free-flowing powder composition, the therapeutic agent is typically formulated with a suitable excipient such as lactose, starch, mannitol, dextrose, polylactic acid (PLA), polylactide-co-glycolide (PLGA) or combinations thereof. Typically, the therapeutic agent is micronized and combined with a suitable carrier to form a composition suitable for inhalation.

A representative pharmaceutical composition for use in a dry powder inhaler comprises lactose and a crystalline solid form of the present disclosure in micronized form. Such a dry powder composition can be made, for example, by combining dry milled lactose with the therapeutic agent and then dry blending the components. The composition is then typically loaded into a dry powder dispenser, or into inhalation cartridges or capsules for use with a dry powder delivery device.

Dry powder inhaler delivery devices suitable for administering therapeutic agents by inhalation are described in the art and examples of such devices are commercially available. For example, representative dry powder inhaler delivery devices or products include Aeolizer (Novartis); Airmax (IVAX); ClickHaler (Innovata Biomed); Diskhaler (GlaxoSmithKline); Diskus/Accuhaler (GlaxoSmithKline); Ellipta (GlaxoSmithKline); Easyhaler (Orion Pharma); Eclipse (Aventis); FlowCaps (Hovione); Handihaler (Boehringer Ingelheim); Pulvinal (Chiesi); Rot ahaler (GlaxoSmithKline); SkyeHaler/Certihaler (SkyePharma); Twisthaler (Schering-Plough); Turbuhaler (AstraZeneca); Ultrahaler (Aventis); and the like.

In another particular embodiment, the pharmaceutical composition is administered by inhalation using a metered-dose inhaler. Such metered-dose inhalers typically discharge a measured amount of a therapeutic agent using a compressed propellant gas. Accordingly, pharmaceutical compositions administered using a metered-dose inhaler typically comprise a solution or suspension of the therapeutic agent in a liquefied propellant. Any suitable liquefied propellant may be employed including hydrofluoroalkanes (HFAs), such as 1,1,1,2-tetrafluoroethane (HFA 134a) and 1,1,1,2,3,3,3-heptafluoro-n-propane, (HFA 227); and chlorofluorocarbons, such as CCl₃F. In a particular embodiment, the propellant is hydrofluoroalkanes. In some embodiments, the hydrofluoroalkane formulation contains a co-solvent, such as ethanol or pentane, and/or a surfactant, such as sorbitan trioleate, oleic acid, lecithin, and glycerin.

A representative pharmaceutical composition for use in a metered-dose inhaler comprises from about 0.01% to about 5% by weight of the crystalline form of the present disclosure; from about 0% to about 20% by weight ethanol; and from about 0% to about 5% by weight surfactant; with the remainder being an HFA propellant. Such compositions are typically prepared by adding chilled or pressurized hydrofluoroalkane to a suitable container containing the crystalline form of this disclosure, ethanol (if present) and the surfactant (if present). To prepare a suspension, the crystalline form of the present disclosure may be micronized and then combined with the propellant. The composition is then loaded into an aerosol canister, which typically forms a portion of a metered-dose inhaler device.

Metered-dose inhaler devices suitable for administering therapeutic agents by inhalation are described in the art and examples of such devices are commercially available. For example, representative metered-dose inhaler devices or products include AeroBid Inhaler System (Forest Pharmaceuticals); Atrovent Inhalation Aerosol (Boehringer Ingelheim); Flovent (GlaxoSmithKline); Maxair Inhaler (3M); Proventil Inhaler (Schering); Serevent Inhalation Aerosol (GlaxoSmithKline); and the like.

In another particular aspect, the pharmaceutical composition is administered by inhalation using a nebulizer inhaler. Such nebulizer devices typically produce a stream of high velocity air that causes the pharmaceutical composition to spray as a mist that is carried into the patient's respiratory tract. Accordingly, when formulated for use in a nebulizer inhaler, the crystalline form of the present disclosure can be dissolved in a suitable carrier to form a solution. Alternatively, the crystalline form of the present disclosure can be micronized or nanomilled and combined with a suitable carrier to form a suspension.

A representative pharmaceutical composition for use in a nebulizer inhaler comprises a solution or suspension comprising from about 0.05 μg/mL to about 20 mg/mL of the crystalline form of the present disclosure and excipients compatible with nebulized formulations. In one embodiment, the solution has a pH of about 3 to about 8.

Nebulizer devices suitable for administering therapeutic agents by inhalation are described in the art and examples of such devices are commercially available. For example, representative nebulizer devices or products include the Respimat Softmist Inhaler (Boehringer Ingelheim); the AERx Pulmonary Delivery System (Aradigm Corp.); the PARI LC Plus Reusable Nebulizer (Pari GmbH); and the like.

In yet another aspect, the pharmaceutical compositions of the present disclosure may alternatively be prepared in a dosage form intended for oral administration. Suitable pharmaceutical compositions for oral administration may be in the form of capsules, tablets, pills, lozenges, cachets, dragees, powders, granules; or as a solution or a suspension in an aqueous or non-aqueous liquid; or as an oil-in-water or water-in-oil liquid emulsion; or as an elixir or syrup; and the like; each containing a predetermined amount of a compound of the present disclosure as an active ingredient.

When intended for oral administration in a solid dosage form, the pharmaceutical compositions of the present disclosure will typically comprise the active agent and one or more pharmaceutically-acceptable carriers, such as sodium citrate or dicalcium phosphate. Optionally or alternatively, such solid dosage forms may also comprise: fillers or extenders, binders, humectants, solution retarding agents, absorption accelerators, wetting agents, absorbents, lubricants, coloring agents, and buffering agents. Release agents, wetting agents, coating agents, sweetening, flavoring and perfuming agents, preservatives and antioxidants can also be present in the pharmaceutical compositions of the present disclosure.

The crystalline solid form may also be formulated as a sterile aqueous suspension or solution for ocular injection. Useful excipients that may be included in such an aqueous formulation include polysorbate 80, carboxymethylcellulose, potassium chloride, calcium chloride, magnesium chloride, sodium acetate, sodium citrate, histidine, α-α-trehalose dihydrate, sucrose, polysorbate 20, hydroxypropyl-β-cyclodextrin, and sodium phosphate. Benzyl alcohol may serve as a preservative and sodium chloride may be included to adjust tonicity. In addition, hydrochloric acid and/or sodium hydroxide may be added to the solution for pH adjustment. Aqueous formulations for ocular injection may be prepared as preservative-free.

Alternative formulations may also include controlled release formulations, liquid dosage forms for oral administration, transdermal patches, and parenteral formulations. Conventional excipients and methods of preparation of such alternative formulations are described, for example, in the reference by Remington, supra.

The following non-limiting examples illustrate representative pharmaceutical compositions of the present disclosure.

Dry Powder Composition

A micronized solid form of the present disclosure (1 g) is blended with milled lactose (25 g). This blended mixture is then loaded into individual blisters of a peelable blister pack in an amount sufficient to provide between about 0.1 mg to about 4 mg of the compound of formula I per dose. The contents of the blisters are administered using a dry powder inhaler.

Dry Powder Composition

A micronized solid form of the present disclosure (1 g) is blended with milled lactose (20 g) to form a bulk composition having a weight ratio of compound to milled lactose of 1:20. The blended composition is packed into a dry powder inhalation device capable of delivering between about 0.1 mg to about 4 mg of the compound of formula I per dose.

Metered-Dose Inhaler Composition

A micronized solid form of the present disclosure (10 g) is dispersed in a solution prepared by dissolving lecithin (0.2 g) in demineralized water (200 mL). The resulting suspension is spray dried and then micronized to form a micronized composition comprising particles having a mean diameter less than about 1.5 μm. The micronized composition is then loaded into metered-dose inhaler cartridges containing pressurized 1,1,1,2-tetrafluoroethane in an amount sufficient to provide about 0.1 mg to about 25 mg of the compound of formula I per dose when administered by the metered dose inhaler.

Nebulizer Composition

A solid form of the present disclosure is added to water, followed by addition of sodium hydroxide and hydrochloric acid to adjust the pH to 3.5 to 5.5 and 3% by weight of glycerol. The solution is stirred well until all the components are dissolved. The solution is administered using a nebulizer device that provides about 0.1 mg to about 25 mg of the compound of formula I per dose.

Aqueous Formulation for Ocular Injection

Each mL of a sterile aqueous suspension includes from 5 mg to 50 mg of the solid form of the present disclosure, sodium chloride for tonicity, 0.99% (w/v) benzyl alcohol as a preservative, 0.75% carboxymethylcellulose sodium, and 0.04% polysorbate. Sodium hydroxide or hydrochloric acid may be included to adjust pH to 5 to 7.5.

Aqueous Formulation for Ocular Injection

A sterile preservative-free aqueous suspension includes from 5 mg/mL to 50 mg/mL of the solid form of the present disclosure in 10 mM sodium phosphate, 40 mM sodium chloride, 0.03% polysorbate 20, and 5% sucrose.

Methods

The present compound, 5-ethyl-2-fluoro-4-(3-(5-(1-methylpiperidin-4-yl)-4,5,6,7-tetrahydro-1H-imidazo[4,5-c]pyridin-2-yl)-1H-indazol-6-yl)phenol, (compound 1), has been shown to be a potent inhibitor of the JAK family of enzymes: JAK1, JAK2, JAK3, and TYK2. Compound 1 has demonstrated potent inhibition of pro-inflammatory and pro-fibrotic cytokines and significant lung retention and therefore compound 1 is useful for the treatment of inflammatory and fibrotic disease of the respiratory tract. Further, it has been recognized that the broad anti-inflammatory effect of JAK inhibitors could suppress normal immune cell function, potentially leading to increased risk of infection. The present compound has been optimized to limit absorption from the lung into the plasma, thus minimizing the risk of immunosuppression.

The anti-inflammatory activity of JAK inhibitors has been robustly demonstrated in preclinical models of asthma (Malaviya et al., Int. Immunopharmacol., 2010, 10, 829-836; Matsunaga et al., Biochem. and Biophys. Res. Commun., 2011, 404, 261-267; Kudlacz et al., Eur. J. Pharmacol, 2008, 582, 154-161). Cytokines implicated in asthma inflammation which signal through the JAK-STAT pathway include IL-2, IL-3, IL-4, IL-5, IL-6, IL-9, IL-11, IL-13, IL-23, IL-31, IL-27, thymic stromal lymphopoietin (TSLP), interferon-γ (IFNγ) and granulocyte-macrophage colony-stimulating factor (GM-CSF). Accordingly, the crystalline form of the present disclosure is expected to be useful for the treatment of inflammatory respiratory disorders, in particular, asthma. Asthma has been classified as “Th2 low” and “Th2 high” subtypes (Simpson et al, Respirology, 2006, 11, 54-61). IL-4, IL-13, IL-5, and TSLP are implicated in Th2 high asthma, while IL-23/IL-12, IL-6, IL-27, and IFNgamma are implicated in Th2 low asthma. Based on its pan JAK inhibitory profile, compound 1 potently inhibits mediators of both Th2 high and Th2 low asthma. It is therefore expected that the crystalline form of the present disclosure will be useful in the treatment of both Th2 high and Th2 low asthma.

Inflammation and fibrosis of the lung is characteristic of other respiratory diseases in addition to asthma such as chronic obstructive pulmonary disease (COPD), cystic fibrosis (CF), pneumonitis, interstitial lung diseases (including idiopathic pulmonary fibrosis), acute lung injury, acute respiratory distress syndrome, bronchitis, emphysema, bronchiolitis obliterans, and sarcoidosis. The crystalline form of the present disclosure is also expected to be useful for the treatment of chronic obstructive pulmonary disease, cystic fibrosis, pneumonitis, interstitial lung diseases (including idiopathic pulmonary fibrosis), acute lung injury, acute respiratory distress syndrome, bronchitis, emphysema, bronchiolitis obliterans, and sarcoidosis.

Compound 1 has demonstrated inhibition of cytokines associated with inflammation. Therefore, the crystalline form of the present disclosure is expected to be useful for the treatment of certain specific respiratory diseases, as detailed below.

Eosinophilic airway inflammation is a characteristic feature of diseases collectively termed eosinophilic lung diseases (Cottin et al., Clin. Chest. Med., 2016, 37(3), 535-56). Eosinophilic diseases have been associated with IL-4, IL-13 and IL-5 signaling. Eosinophilic lung diseases include infections (especially helminthic infections), drug-induced pneumonitis (induced for example by therapeutic drugs such as antibiotics, phenytoin, or 1-tryptophan), fungal-induced pneumonitis (e.g. allergic bronchopulmonary aspergillosis), hypersensitivity pneumonitis and eosinophilic granulomatosis with polyangiitis (formerly known as Churg-Strauss syndrome). Eosinophilic lung diseases of unknown etiology include idiopathic acute eosinophilic pneumonia, idiopathic chronic eosinophilic pneumonia, hypereosinophilic syndrome, and Löffler syndrome.

A polymorphism in the IL-6 gene has been associated with elevated IL-6 levels and an increased risk of developing pulmonary arterial hypertension (PAH) (Fang et al., J. Am. Soc. Hypertens., 2017, 11(3), 171-177). Corroborating the role of IL-6 in PAH, inhibition of the IL-6 receptor chain gp130 ameliorated the disease in a rat model of PAH (Huang et al., Can. J. Cardiol., 2016, 32(11), 1356.e1-1356.e10).

Cytokines such as IFNγ, IL-12 and IL-6 have been implicated in a range of non-allergic lung diseases such as sarcoidosis, and lymphangioleiomyomatosis (El-Hashemite et al., Am. J. Respir. Cell. Mol. Biol., 2005, 33, 227-230, and El-Hashemite et al., Cancer Res., 2004, 64, 3436-3443).

Bronchiectasis and infiltrative pulmonary diseases are diseases associated with chronic neutrophilic inflammation.

Pathological T cell activation is critical in the etiology of multiple respiratory diseases. Autoreactive T cells play a role in bronchiolitis obliterans organizing pneumonia (also termed COS). Similar to COS the etiology of lung transplant rejections is linked to an aberrant T cell activation of the recipients T cells by the transplanted donor lung. Lung transplant rejections may occur early as Primary Graft Dysfunction (PGD), organizing pneumonia (OP), acute rejection (AR) or lymphocytic bronchiolitis (LB) or they may occur years after lung transplantation as Chronic Lung Allograft Dysfunction (CLAD). CLAD was previously known as bronchiolitis obliterans (BO) but now is considered a syndrome that can have different pathological manifestations including BO, restrictive CLAD (rCLAD or RAS) and neutrophilic allograft dysfunction. Chronic lung allograft dysfunction (CLAD) is a major challenge in long-term management of lung transplant recipients as it causes a transplanted lung to progressively lose functionality (Gauthier et al., Curr Transplant Rep., 2016, 3(3), 185-191). CLAD is poorly responsive to treatment and therefore, there remains a need for effective compounds capable of preventing or treating this condition. Several JAK-dependent cytokines such as IFNγ and IL-5 are up-regulated in CLAD and lung transplant rejection (Berastegui et al, Clin. Transplant. 2017, 31, e12898). Moreover, high lung levels of CXCR3 chemokines such as CXCL9 and CXCL10 which are downstream of JAK-dependent IFN signaling, are linked to worse outcomes in lung transplant patients (Shino et al, PLOS One, 2017, 12 (7), e0180281). Systemic JAK inhibition has been shown to be effective in kidney transplant rejection (Vicenti et al., American Journal of Transplantation, 2012, 12, 2446-56). Therefore, JAK inhibitors have the potential to be effective in preventing or delaying lung transplant rejection and CLAD. Similar T cell activation events as described as the basis for lung transplant rejection also are considered the main driver of lung graft-versus-host disease (GVHD) which can occur post hematopoietic stem cell transplants. Similar to CLAD, lung GVHD is a chronic progressive condition with extremely poor outcomes and no treatments are currently approved. A retrospective, multicenter survey study of 95 patients with steroid-refractory acute or chronic GVHD who received the systemic JAK inhibitor ruxolitinib as salvage therapy demonstrated complete or partial response to ruxolitinib in the majority of patients including those with lung GVHD (Zeiser et al, Leukemia, 2015, 29, 10, 2062-68). As systemic JAK inhibition is associated with serious adverse events and a small therapeutic index, the need remains for an inhaled lung-directed, non-systemic JAK inhibitor to prevent or delay lung transplant rejection or lung GVHD. Compound 1 and the crystalline form of the present disclosure have the characteristics required to meet this need. More recently, immune-checkpoint inhibitor induced pneumonitis, another T cell mediated lung disease emerged with the increased use of immune-checkpoint inhibitors. In cancer patients treated with these T cell stimulating agents, fatal pneumonitis can develop.

In one embodiment, therefore, the present disclosure provides a method of treating a respiratory disease in a mammal (e.g., a human), the method comprising administering to the mammal (or human) a therapeutically-effective amount of the crystalline form of the present disclosure, or of a pharmaceutical composition comprising the crystalline form of the present disclosure and a pharmaceutically-acceptable carrier.

In one embodiment, the respiratory disease is selected from the group consisting of asthma, chronic obstructive pulmonary disease, cystic fibrosis, pneumonitis, idiopathic pulmonary fibrosis, acute lung injury, acute respiratory distress syndrome, bronchitis, emphysema, sarcoidosis, an eosinophilic disease, a lung infection, a helminthic infection, pulmonary arterial hypertension, lymphangioleiomyomatosis, bronchiectasis, an infiltrative pulmonary disease, drug-induced pneumonitis, fungal induced pneumonitis, allergic bronchopulmonary aspergillosis, hypersensitivity pneumonitis, eosinophilic granulomatosis with polyangiitis, idiopathic acute eosinophilic pneumonia, idiopathic chronic eosinophilic pneumonia, hypereosinophilic syndrome, Löffler syndrome, bronchiolitis obliterans organizing pneumonia, lung graft-versus-host disease, and immune-checkpoint-inhibitor induced pneumonitis. In some embodiments, the respiratory disease is asthma. In some embodiments, the asthma is moderate to severe asthma. In some embodiments, the asthma is mild to moderate asthma. In some embodiments, the pharmaceutical composition is administered by inhalation. In some embodiments, the asthma is Th2 high asthma. In some embodiments, the asthma is Th2 low asthma.

In one embodiment, the present disclosure provides a method of preventing or delaying lung transplant rejection in a mammal (e.g., a human), the method comprising administering to the mammal (or human) a therapeutically-effective amount of the crystalline form of the present disclosure, or of a pharmaceutical composition comprising the crystalline form of the present disclosure and a pharmaceutically-acceptable carrier. In some embodiments, the lung transplant rejection is selected from the group consisting of primary graft dysfunction, organizing pneumonia, acute rejection, lymphocytic bronchiolitis, and chronic lung allograft dysfunction. In some embodiments, the lung transplant rejection is acute lung transplant rejection. In some embodiments, the lung transplant rejection is chronic lung allograft dysfunction. In some embodiments, the lung transplant rejection is selected from the group consisting of bronchiolitis obliterans, restrictive chronic lung allograft dysfunction, and neutrophilic allograft dysfunction. In some embodiments, the crystalline form of the present disclosure or the corresponding pharmaceutical composition is administered by inhalation.

Also provided herein are uses of the crystalline form of the present disclosure in medical therapy and in the manufacture of a formulation or medicament for treating, preventing, delaying, or ameliorating diseases amenable to treatment with a JAK inhibitor, in particular respiratory diseases and lung transplant rejection.

The present disclosure further provides a method of treating asthma in a mammal, the method comprising administering to the mammal a therapeutically-effective amount of the crystalline form of the present disclosure, or of a pharmaceutical composition comprising a pharmaceutically-acceptable carrier and the crystalline form of the present disclosure.

When used to treat asthma, the crystalline form of the present disclosure, will typically be administered in a single daily dose or in multiple doses per day, although other forms of administration may be used. The amount of active agent administered per dose or the total amount administered per day will typically be determined by a physician, in the light of the relevant circumstances, including the condition to be treated, the chosen route of administration, the actual compound administered and its relative activity, the age, weight, and response of the individual patient, the severity of the patient's symptoms, and the like.

The present disclosure further provides a method of treating a respiratory disease (including but not limited to the disease described herein) in a mammal, the method comprising administering to the mammal a therapeutically-effective amount of the crystalline form of the present disclosure, or of a pharmaceutical composition comprising a pharmaceutically-acceptable carrier and the crystalline form of the present disclosure.

Human coronavirus is a common respiratory pathogen and typically induces mild upper respiratory disease. The two highly pathogenic viruses, Severe Acute Respiratory Syndrome associated-Coronavirus (SARS-CoV-1) and Middle East Respiratory Syndrome-associated Coronavirus (MERS-CoV), caused severe respiratory syndromes resulting in more than 10% and 35% mortality, respectively (Assiri et al., N Engl J Med., 2013, 369, 407-1). The recent emergence of Coronavirus Disease 2019 (COVID-19 and subsequent pandemic has created a global health care emergency. Similar to SARS-CoV-1 and MERS-CoV, a subset of patients (about 16%) can develop a severe respiratory illness manifested by acute lung injury (ALI) leading to ICU admission (about 5%), respiratory failure (about 6.1%) and death (Wang et al., JAMA, 2020, 323, 11, 1061-1069; Guan et al., N Engl J Med., 2020, 382, 1708-1720; Huang et al., The Lancet, 2020. 395 (10223), 497-506; Chen et al., The Lancet, 2020, 395(10223), 507-13). A subgroup of patients with COVID-19 appears to have a hyperinflammatory “cytokine storm” resulting in acute lung injury and acute respiratory distress syndrome (ARDS). This cytokine storm may also spill over into the systemic circulation and produce sepsis and ultimately, multi-organ dysfunction syndrome. The dysregulated cytokine signaling that appears in COVID-19 is characterized by increased expression of interferons (IFNs), interleukins (ILs), and chemokines, resulting in ALI and associated mortality. This hyperinflammatory response can potentially be modulated and treated by a lung-selective pan-Janus Kinase (JAK) inhibitor. Though in-vivo models of COVID-19 have yet to be published, infection with mouse adapted strains of the 2003 SARS-CoV-1 and 2012 MERS-CoV, as well as a transgenic mouse expressing the human SARS-CoV-1 receptor hACE2 infected with human SARS-CoV-1, demonstrate elevations of JAK-dependent cytokines, such as IFNγ, IL-6, and IL-12, and downstream chemokines, such as chemokine (C-C motif) ligand 10 (CCL10), CCL2, and CCL7 (McCray et al., J Virol., 2007, 81(2), 813-21; Gretebeck et al., Curr Opin Virol. 2015, 13, 123-9.; Day et al., Virology. 2009, 395(2), 210-22. JAK inhibitors have also been shown to be beneficial in mouse models of lipopolysaccharide- or ganciclovir-induced ALI (Severgnini et al., Am J Respir Crit Care Med., 2005, 171(8), 858-67; Jin et al., Am J Physiol-Lung Cell Mol Physiol., 2018, 314(5), L882-92).

Therefore, the crystalline form of the present disclosure may be useful to dampen the cytokine storm associated with COVID-19. By delivering to the lung and avoiding systemic immunosuppression, additional infections that lead to worsened mortality may also be avoided. This is particularly true in those patients requiring ventilatory support. As major causes of death in subjects with COVID-19 appear to be comorbidities and superinfection, an inhaled medication may be a way to avoid systemic immunosuppression that would pre-dispose patients to these risks.

Therefore, the present disclosure provides a method of treating a mammal (or patient) infected with a coronavirus such as SARS-CoV-1, SARS-CoV-2, and MERS-CoV, or the symptoms thereof, the method comprising administering to the mammal (or patient) the crystalline form of the present disclosure, or a pharmaceutical composition comprising a pharmaceutically-acceptable carrier and the crystalline form of the present disclosure. The present disclosure also provides a method of treating ALI and/or ARDS in a mammal (or a patient) caused by a coronavirus infection (such as SARS-CoV-1, SARS-CoV-2, and MERS-CoV), the method comprising administering to the mammal (or patient) the crystalline form of the present disclosure, or a pharmaceutical composition comprising a pharmaceutically-acceptable carrier and the crystalline form of the present disclosure.

The mechanism of action of JAK inhibitors has been linked to the treatment of nasal inflammatory diseases (Therapeutic Effects of Intranasal Tofacitinib on Chronic Rhinosinusitis with Nasal Polyps in Mice, Joo et al., The Laryngoscope, 2020, haps://doi.org/10.1002/lary.29129). Further, Dupilumab, which acts by blocking the IL-4 and IL-13 signaling pathways, has been approved for the treatment of chronic rhinosinusitis with nasal polyps. Therefore, also provided herein is a method of treating nasal inflammatory diseases in a mammal (e.g. a human), the method comprising administering to the mammal (or human) the crystalline form of the present disclosure, or a pharmaceutical composition comprising a pharmaceutically-acceptable carrier and the crystalline form of the present disclosure. In some embodiments, the nasal inflammatory disease is selected from the group consisting of chronic rhinosinusitis with or without nasal polyps, nasal polyposis, sinusitis with nasal polyps, and rhinitis (non-allergic, allergic, perenial, and vasomotor rhinitis).

As a JAK inhibitor, the crystalline form of the present disclosure may also be useful for a variety of other diseases. The crystalline form of the present disclosure may be useful for a variety of gastrointestinal inflammatory indications that include, but are not limited to, inflammatory bowel disease, ulcerative colitis (proctosigmoiditis, pancolitis, ulcerative proctitis and left-sided colitis), Crohn's disease, collagenous colitis, lymphocytic colitis, Behcet's disease, celiac disease, immune checkpoint inhibitor induced colitis, ileitis, eosinophilic esophagitis, graft versus host disease-related colitis, and infectious colitis. Ulcerative colitis (Reimund et al., J. Clin. Immunology, 1996, 16, 144-150), Crohn's disease (Woywodt et al., Eur. J. Gastroenterology Hepatology, 1999, 11, 267-276), collagenous colitis (Kumawat et al., Mol. Immunology, 2013, 55, 355-364), lymphocytic colitis (Kumawat et al., 2013), eosinophilic esophagitis (Weinbrand-Goichberg et al., Immunol. Res., 2013, 56, 249-260), graft versus host disease-related colitis (Coghill et al., Blood, 2001, 117, 3268-3276), infectious colitis (Stallmach et al., Int. J. Colorectal Dis., 2004, 19, 308-315), Behcet's disease (Zhou et al., Autoimmun. Rev., 2012, 11, 699-704), celiac disease (de Nitto et al., World J. Gastroenterol., 2009, 15, 4609-4614), immune checkpoint inhibitor induced colitis (e.g., CTLA-4 inhibitor-induced colitis; (Yano et al., J. Translation. Med., 2014, 12, 191), PD-1- or PD-L1-inhibitor-induced colitis), and ileitis (Yamamoto et al., Dig. Liver Dis., 2008, 40, 253-259) are characterized by elevation of certain pro-inflammatory cytokine levels. As many pro-inflammatory cytokines signal via JAK activation, the crystalline form of the present disclosure may be able to alleviate the inflammation and provide symptom relief. In particular, the crystalline form of the present disclosure may be useful for the induction and maintenance of remission of ulcerative colitis, and for the treatment of Crohn's disease, immune checkpoint inhibitor induced colitis, and the gastrointestinal adverse effects in graft versus host disease. In one embodiment, therefore, the disclosure provides a method of treating a gastrointestinal inflammatory disease in a mammal (e.g., a human), the method comprising administering to the mammal the crystalline form of the present disclosure, or a pharmaceutical composition comprising a pharmaceutically-acceptable carrier and the crystalline form of the present disclosure.

Atopic dermatitis and other inflammatory skin diseases have been associated with elevation of proinflammatory cytokines that rely on the JAK-STAT pathway. Therefore, the crystalline form of the present disclosure, may be beneficial in a number of dermal inflammatory or pruritic conditions that include, but are not limited to atopic dermatitis, alopecia areata, vitiligo, psoriasis, dermatomyositis, cutaneous T cell lymphoma (Netchiporouk et al., Cell Cycle 2014; 13, 3331-3335) and subtypes (Sezary syndrome, mycosis fungoides, pagetoid reticulosis, granulomatous slack skin, lymphomatoid papulosis, Pityriasis lichenoides chronica, Pityriasis lichenoides et Varioliformis acuta, CD30+ cutaneous T-cell lymphoma, secondary cutaneous CD30+ large cell lymphoma, non-mycosis fungoides CD30− cutaneous large T-cell lymphoma, pleomorphic T-cell lymphoma, Lennert lymphoma, subcutaneous T-cell lymphoma, angiocentric lymphoma, blastic NK-cell lymphoma), prurigo nodularis, lichen planus, primary localized cutaneous amyloidosis, bullous pemphigoid, skin manifestations of graft versus host disease, pemphigoid, discoid lupus, granuloma annulare, lichen simplex chronicus, vulvar/scrotal/perianal pruritus, lichen sclerosus, post herpetic neuralgia itch, lichen planopilaris, and foliculitis decalvans. In particular, atopic dermatitis (Bao et al., JAK-STAT, 2013, 2, e24137), alopecia areata (Xing et al., Nat. Med. 2014, 20, 1043-1049), vitiligo (Craiglow et al, JAMA Dermatol. 2015, 151, 1110-1112), prurigo nodularis (Sonkoly et al., J. Allergy Clin. Immunol. 2006, 117, 411-417), lichen planus (Welz-Kubiak et al., J. Immunol. Res. 2015, ID:854747), primary localized cutaneous amyloidosis (Tanaka et al., Br. J. Dermatol. 2009, 161, 1217-1224), bullous pemphigoid (Feliciani et al., Int. J. Immunopathol. Pharmacol. 1999, 12, 55-61), and dermal manifestations of graft versus host disease (Okiyama et al., J. Invest. Dermatol. 2014, 134, 992-1000) are characterized by elevation of certain cytokines that signal via JAK activation. Accordingly, the crystalline form of the present disclosure, may be able to alleviate associated dermal inflammation or pruritus driven by these cytokines. In particular, the crystalline form of the present disclosure, may be expected to be useful for the treatment of atopic dermatitis and other inflammatory skin diseases. In one embodiment, therefore, the disclosure provides a method of treating an inflammatory skin disease in a mammal (e.g., a human), the method comprising applying a pharmaceutical composition comprising the crystalline form of the present disclosure, and a pharmaceutical carrier to the skin of the mammal. In one embodiment, the inflammatory skin disease is atopic dermatitis.

Many ocular diseases have been shown to be associated with elevations of proinflammatory cytokines that rely on the JAK-STAT pathway. The crystalline form of the present disclosure, therefore, may be useful for the treatment of a number of ocular diseases that include, but are not limited to, uveitis, diabetic retinopathy, diabetic macular edema, dry eye disease, age-related macular degeneration, and atopic keratoconjunctivitis. In particular, uveitis (Horai and Caspi, J. Interferon Cytokine Res., 2011, 31, 733-744), diabetic retinopathy (Abcouwer, J. Clin. Cell. Immunol., 2013, Suppl 1, 1-12), diabetic macular edema (Sohn et al., American Journal of Ophthalmology, 2011, 152, 686-694), dry eye disease (Stevenson et al, Arch. Ophthalmol., 2012, 130, 90-100), and age-related macular degeneration (Knickelbein et al, Int. Ophthalmol. Clin., 2015, 55(3), 63-78) are characterized by elevation of certain pro-inflammatory cytokines that signal via the JAK-STAT pathway. Retinal vein occlusion (RVO) is a highly prevalent visually disabling disease. Obstruction of retinal blood flow can lead to damage of the retinal vasculature, hemorrhage, and tissue ischemia. Although the causes for RVO are multifactorial, both vascular as well as inflammatory mediators have been shown to be important (Deobhakta et al, International Journal of Inflammation, 2013, article ID 438412). Cytokines which signal through the JAK-STAT pathway, such as IL-6 and IL-13, as well as other cytokines, such as MCP-1, whose production is driven in part by JAK-STAT pathway signaling, have been detected at elevated levels in ocular tissues of patients with RVO (Shchuko et al, Indian Journal of Ophthalmology, 2015, 63(12), 905-911). Accordingly, the crystalline form of the present disclosure may be able to alleviate the associated ocular inflammation and reverse disease progression or provide symptom relief in this disease. While many patients with RVO are treated by photocoagulation, this is an inherently destructive therapy. Anti-VEGF agents are also used, but they are only effective in a fraction of patients. Steroid medications that reduce the level of inflammation in the eye (Triamcinolone acetonide and dexamethasone implants) have also been shown to provide beneficial results for patients with certain forms of RVO, but they have also been shown to cause cataracts and increased intraocular pressure/glaucoma.

Accordingly, the crystalline form of the present disclosure may be able to alleviate the associated ocular inflammation and reverse disease progression or provide symptom relief. In one embodiment, therefore, the disclosure provides a method of treating an ocular disease in a mammal, the method comprising administering a pharmaceutical composition comprising the crystalline form of the present disclosure and a pharmaceutical carrier to the eye of the mammal. In one embodiment, the ocular disease is uveitis, diabetic retinopathy, diabetic macular edema, dry eye disease, age-related macular degeneration, retinal vein occlusion or atopic keratoconjunctivitis. In one embodiment, the method comprises administering the crystalline form of the present disclosure by intravitreal injection. The crystalline form of the present disclosure may also be used in combination with one or more compound useful to ocular diseases.

The crystalline form of the present disclosure may also be useful to treat other diseases such as other inflammatory diseases, autoimmune diseases or cancers. The crystalline form of the present disclosure may be useful to treat one or more of arthritis, rheumatoid arthritis, juvenile rheumatoid arthritis, transplant rejection, xerophthalmia, psoriatic arthritis, diabetes, insulin dependent diabetes, motor neurone disease, myelodysplastic syndrome, pain, sarcopenia, cachexia, septic shock, systemic lupus erythematosus, leukemia, chronic lymphocytic leukemia, chronic myelocytic leukemia, acute lymphoblastic leukemia, acute myelogenous leukemia, ankylosing spondylitis, myelofibrosis, B-cell lymphoma, hepatocellular carcinoma, Hodgkins disease, breast cancer, Multiple myeloma, melanoma, non-Hodgkin lymphoma, non-small-cell lung cancer, ovarian clear cell carcinoma, ovary tumor, pancreas tumor, polycythemia vera, Sjogren's syndrome, soft tissue sarcoma, sarcoma, splenomegaly, T-cell lymphoma, and thalassemia major.

Combination Therapy

The crystalline form of the present disclosure may be used in combination with one or more agents which act by the same mechanism or by different mechanisms to treat a disease. The different agents may be administered sequentially or simultaneously, in separate compositions or in the same composition. Useful classes of agents for combination therapy include, but are not limited to, a beta 2 adrenoceptor agonist, a muscarinic receptor antagonist, a glucocorticoid agonist, a G-protein coupled receptor-44 antagonist, a leukotriene D4 antagonist, a muscarinic M3 receptor antagonist, a histamine H1 receptor antagonist, an immunoglobulin E antagonist, a PDE 4 inhibitor, an IL-4 antagonist, a muscarinic M1 receptor antagonist, a histamine receptor antagonist, an IL-13 antagonist, an IL-5 antagonist, a 5-Lipoxygenase inhibitor, a beta adrenoceptor agonist, a CCR3 chemokine antagonist, a CFTR stimulator, an immunoglobulin modulator, an interleukin 33 ligand inhibitor, a PDE 3 inhibitor, a phosphoinositide-3 kinase delta inhibitor, a thromboxane A2 antagonist, an elastase inhibitor, a Kit tyrosine kinase inhibitor, a leukotriene E4 antagonist, a leukotriene antagonist, a PGD2 antagonist, a TNF alpha ligand inhibitor, a TNF binding agent, a complement cascade inhibitor, an eotaxin ligand inhibitor, a glutathione reductase inhibitor, an histamine H4 receptor antagonist, an IL-6 antagonist, an IL2 gene stimulator, an immunoglobulin gamma Fc receptor IIB modulator, an interferon gamma ligand, an interleukin 13 ligand inhibitor, an interleukin 17 ligand inhibitor, a L-Selectin antagonist, a leukocyte elastase inhibitor, a leukotriene C4 antagonist, a Leukotriene C4 synthase inhibitor, a membrane copper amine oxidase inhibitor, a metalloprotease-12 inhibitor, a metalloprotease-9 inhibitor, a mite allergen modulator, a muscarinic receptor modulator, a nicotinic acetylcholine receptor agonist, a nuclear factor kappa B inhibitor, a p-Selectin antagonist, a PDE 5 inhibitor, a PDGF receptor antagonist, a phosphoinositide-3 kinase gamma inhibitor, a TLR-7 agonist, a TNF antagonist, an Abl tyrosine kinase inhibitor, an acetylcholine receptor antagonist, an acidic mammalian chitinase inhibitor, an ACTH receptor agonist, an actin polymerization modulator, an adenosine A1 receptor antagonist, an adenylate cyclase stimulator, an adrenoceptor antagonist, an adrenocorticotrophic hormone ligand, an alcohol dehydrogenase 5 inhibitor, an alpha 1 antitrypsin stimulator, an alpha 1 proteinase inhibitor, an androgen receptor modulator, an angiotensin converting enzyme 2 stimulator, an ANP agonist, a Bcr protein inhibitor, a beta 1 adrenoceptor antagonist, a beta 2 adrenoceptor antagonist, a beta 2 adrenoceptor modulator, a beta amyloid modulator, a BMP10 gene inhibitor, a BMP15 gene inhibitor, a calcium channel inhibitor, a cathepsin G inhibitor, a CCL26 gene inhibitor, a CCR3 chemokine modulator, a CCR4 chemokine antagonist, a cell adhesion molecule inhibitor, a chaperonin stimulator, a chitinase inhibitor, a collagen I antagonist, a complement C3 inhibitor, a CSF-1 antagonist, a CXCR2 chemokine antagonist, a cytokine receptor common beta chain modulator, a cytotoxic T-lymphocyte protein-4 stimulator, a deoxyribonuclease I stimulator, a deoxyribonuclease stimulator, a dipeptidyl peptidase I inhibitor, a DNA gyrase inhibitor, a DP prostanoid receptor modulator, an E-Selectin antagonist, an EGFR family tyrosine kinase receptor inhibitor, an elastin modulator, an Endothelin ET-A antagonist, an Endothelin ET-B antagonist, an epoxide hydrolase inhibitor, a FGF3 receptor antagonist, a Fyn tyrosine kinase inhibitor, a GATA 3 transcription factor inhibitor, a Glucosylceramidase modulator, a Glutamate receptor modulator, a GM-CSF ligand inhibitor, a Guanylate cyclase stimulator, a H+ K+ ATPase inhibitor, an hemoglobin modulator, an Heparin agonist, an Histone deacetylase inhibitor, an Histone deacetylase-2 stimulator, an HMG CoA reductase inhibitor, an I-kappa B kinase beta inhibitor, an ICAM1 gene inhibitor, an IL-17 antagonist, an IL-17 receptor modulator, an IL-23 antagonist, an IL-4 receptor modulator, an Immunoglobulin G modulator, an Immunoglobulin G1 agonist, an Immunoglobulin G1 modulator, an Immunoglobulin epsilon Fc receptor IA antagonist, an Immunoglobulin gamma Fc receptor IIB antagonist, an Immunoglobulin kappa modulator, an Insulin sensitizer, an Interferon beta ligand, an Interleukin 1 like receptor antagonist, an Interleukin 18 ligand inhibitor, an Interleukin receptor 17A antagonist, an Interleukin-1 beta ligand inhibitor, an Interleukin-5 ligand inhibitor, an Interleukin-6 ligand inhibitor, a KCNA voltage-gated potassium channel-3 inhibitor, a Kit ligand inhibitor, a Laminin-5 agonist, a Leukotriene CysLT1 receptor antagonist, a Leukotriene CysLT2 receptor antagonist, a LOXL2 gene inhibitor, a Lyn tyrosine kinase inhibitor, a MARCKS protein inhibitor, a MDR associated protein 4 inhibitor, a Metalloprotease-2 modulator, a Metalloprotease-9 modulator, a Mineralocorticoid receptor antagonist, a Muscarinic M2 receptor antagonist, a Muscarinic M4 receptor antagonist, a Muscarinic M5 receptor antagonist, a Natriuretic peptide receptor A agonist, a Natural killer cell receptor modulator, a Nicotinic ACh receptor alpha 7 subunit stimulator, a NK cell receptor modulator, a Nuclear factor kappa B modulator, an opioid growth factor receptor agonist, a P-Glycoprotein inhibitor, a P2X3 purinoceptor antagonist, a p38 MAP kinase inhibitor, a Peptidase 1 modulator, a phospholipase A2 inhibitor, a phospholipase C inhibitor, a plasminogen activator inhibitor 1 inhibitor, a platelet activating factor receptor antagonist, a PPAR gamma agonist, a prostacyclin agonist, a protein tyrosine kinase inhibitor, a SH2 domain inositol phosphatase 1 stimulator, a signal transduction inhibitor, a sodium channel inhibitor, a STAT-3 modulator, a Stem cell antigen-1 inhibitor, a superoxide dismutase modulator, a T cell surface glycoprotein CD28 inhibitor, a T-cell surface glycoprotein CD8 inhibitor, a TGF beta agonist, a TGF beta antagonist, a thromboxane synthetase inhibitor, a thymic stromal lymphoprotein ligand inhibitor, a thymosin agonist, a thymosin beta 4 ligand, a TLR-8 agonist, a TLR-9 agonist, a TLR9 gene stimulator, a Topoisomerase IV inhibitor, a Troponin I fast skeletal muscle stimulator, a Troponin T fast skeletal muscle stimulator, a Type I IL-1 receptor antagonist, a Type II TNF receptor modulator, an ion channel modulator, a uteroglobin stimulator, and a VIP agonist.

Specific agents that may be used in combination with the crystalline form of the present disclosure include, but are not limited to rosiptor acetate, umeclidinium bromide, secukinumab, metenkefalin acetate, tridecactide acetate, fluticasone propionate, alpha-cyclodextrin-stabilized sulforaphane, tezepelumab, mometasone furoate, BI-1467335, dupilumab, aclidinium, formoterol, AZD-1419, HI-1640V, rivipansel, CMP-001, mannitol, ANB-020, omalizumab, tregalizumab, Mitizax, benralizumab, golimumab, roflumilast, imatinib, REGN-3500, masitinib, apremilast, RPL-554, Actimmune, adalimumab, rupatadine, parogrelil, MK-1029, beclometasone dipropionate, formoterol fumarate, mogamulizumab, seratrodast, UCB-4144, nemiralisib, CK-2127107, fevipiprant, danirixin, bosentan, abatacept, EC-18, duvelisib, dociparstat, ciprofloxacin, salbutamol HFA, erdosteine, PrEP-001, nedocromil, CDX-0158, salbutamol, enobosarm, R-TPR-022, lenzilumab, fluticasone furoate, vilanterol trifenatate, fluticasone propionate, salmeterol, PT-007, PRS-060, remestemcel-L, citrulline, RPC-4046, nitric oxide, DS-102, gerilimzumab, Actair, fluticasone furoate, umeclidinium, vilanterol, AG-NPP709, Gamunex, infliximab, Ampion, acumapimod, canakinumab, INS-1007, CYP-001, sirukumab, fluticasone propionate, mepolizumab, pitavastatin, solithromycin, etanercept, ivacaftor, anakinra, MPC-300-IV, glycopyrronium bromide, aclidinium bromide, FP-025, risankizumab, glycopyrronium, formoterol fumarate, Adipocell, YPL-001, tiotropium bromide, glycopyrronium bromide, indacaterol maleate, andecaliximab, olodaterol, esomeprazole, dust mite vaccine, mugwort pollen allergen vaccine, vamorolone, gefapixant, revefenacin, gefitinib, ReJoin, tipelukast, bedoradrine, SCM-CGH, SHP-652, RNS-60, brodalumab, BIO-11006, umeclidinium bromide, vilanterol trifenatate, ipratropium bromide, tralokinumab, PUR-1800, VX-561, VX-371, olopatadine, tulobuterol, formoterol fumarate, triamcinolone acetonide, reslizumab, salmeterol xinafoate, fluticasone propionate, beclometasone dipropionate, formoterol fumarate, tiotropium bromide, ligelizumab, RUTI, bertilimumab, omalizumab, glycopyrronium bromide, SENS-111, beclomethasone dipropionate, CHF-5992, LT-4001, indacaterol, glycopyrronium bromide, mometasone furoate, fexofenadine, glycopyrronium bromide, azithromycin, AZD-7594, formoterol, CHF-6001, batefenterol, OATD-01, olodaterol, CJM-112, rosiglitazone, salmeterol, setipiprant, inhaled interferon beta, AZD-8871, plecanatide, fluticasone, salmeterol, eicosapentaenoic acid monoglycerides, lebrikizumab, RG-6149, QBKPN, Mometasone, indacaterol, AZD-9898, sodium pyruvate, zileuton, CG-201, imidafenacin, CNTO-6785, CLBS-03, mometasone, RGN-137, procaterol, formoterol, CCI-15106, POL-6014, indacaterol, beclomethasone, MV-130, GC-1112, Allergovac depot, MEDI-3506, QBW-251, ZPL-389, udenafil, GSK-3772847, levocetirizine, AXP-1275, ADC-3680, timapiprant, abediterol, AZD-7594, ipratropium bromide, salbutamol sulfate, tadekinig alfa, ACT-774312, dornase alfa, iloprost, batefenterol, fluticasone furoate, alicaforsen, ciclesonide, emeramide, arformoterol, SB-010, Ozagrel, BTT-1023, Dectrekumab, levalbuterol, pranlukast, hyaluronic acid, GSK-2292767, Formoterol, NOV-14, Lucinactant, salbutamol, prednisolone, ebastine, dexamethasone cipecilate, GSK-2586881, BI-443651, GSK-2256294, VR-179, VR-096, hdm-ASIT+, budesonide, GSK-2245035, VTX-1463, Emedastine, dexpramipexole, levalbuterol, N-6022, dexamethasone sodium phosphate, PIN-201104, OPK-0018, TEV-48107, suplatast, BI-1060469, Gemilukast, interferon gamma, dalazatide, bilastine, fluticasone propionate, salmeterol xinafoate, RP-3128, bencycloquidium bromide, reslizumab, PBF-680, CRTH2 antagonist, Pranlukast, salmeterol xinafoate, fluticasone propionate, tiotropium bromide monohydrate, masilukast, RG-7990, Doxofylline, abediterol, glycopyrronium bromide, TEV-46017, ASM-024, fluticasone propionate, glycopyrronium bromide, salmeterol xinafoate, salbutamol, TA-270, Flunisolide, sodium chromoglycate, Epsi-gam, ZPL-521, salbutamol, aviptadil, TRN-157, Zafirlukast, Stempeucel, pemirolast sodium, nadolol, fluticasone propionate+salmeterol xinafoate, RV-1729, salbutamol sulfate, carbon dioxide+perfluorooctyl bromide, APL-1, dectrekumab+VAK-694, lysine acetylsalicylate, zileuton, TR-4, human allogenic adipose-derived mesenchymal progenitor cell therapy, MEDI-9314, PL-3994, HMP-301, TD-5471, NKTT-120, pemirolast, beclomethasone dipropionate, trantinterol, monosodium alpha luminol, IMD-1041, AM-211, TBS-5, ARRY-502, seratrodast, recombinant midismase, ASM-8, deflazacort, bambuterol, RBx-10017609, ipratropium+fenoterol, fluticasone+formoterol, epinastine, WIN-901X, VALERGEN-DS, OligoG-COPD-5/20, tulobuterol, oxis Turbuhaler, DSP-3025, ASM-024, mizolastine, budesonide+salmeterol, LH-011, AXP-E, histamine human immunoglobulin, YHD-001, theophylline, ambroxol+erdosteine, ramatroban, montelukast, pranlukast, AG-1321001, tulobuterol, ipratropium+salbutamol, tranilast, methylprednisolone suleptanate, colforsin daropate, repirinast, and doxofylline.

Also provided, herein, is a pharmaceutical composition comprising the crystalline form of the present disclosure and one or more other therapeutic agents. The therapeutic agent may be selected from the class of agents specified above and from the list of specific agents described above. In some embodiments, the pharmaceutical composition is suitable for delivery to the lungs. In some embodiments, the pharmaceutical composition is suitable for inhaled or nebulized administration. In some embodiments, the pharmaceutical composition is a dry powder, a liquid, or a suspension.

Further, in a method embodiment, the disclosure provides a method of treating a disease or disorder in a mammal comprising administering to the mammal the crystalline form of the present disclosure and one or more other therapeutic agents.

When used in combination therapy, the agents may be formulated in a single pharmaceutical composition, or the agents may be provided in separate compositions that are administered simultaneously or at separate times, by the same or by different routes of administration. Such compositions can be packaged separately or may be packaged together as a kit. The two or more therapeutic agents in the kit may be administered by the same route of administration or by different routes of administration.

### EXAMPLES

The following synthetic and biological examples are illustrative, and are not to be construed in any way as limiting the scope of the disclosure. In the examples below, the following abbreviations have the following meanings unless otherwise indicated. Abbreviations not defined below have their generally accepted meanings.

DPPC=dipalmitoylphosphatidylcholine

DMSO=dimethyl sulfoxide

h=hour(s)

min=minute(s)

RT=room temperature

Reagents and solvents were purchased from commercial suppliers (Aldrich, Fluka, Sigma, etc.), and used without further purification.

Characterization of reaction products was routinely carried out by mass and ¹H-NMR spectrometry. Mass spectrometric identification of compounds was performed by an electrospray ionization method (ESMS) with an Applied Biosystems (Foster City, Calif.) model API 150 EX instrument or a Waters (Milford, Mass.) 3100 instrument, coupled to autopurification systems.

### Preparative HPLC Conditions

### C18, 5 μm. 21.2×150 mm or C18, 5 μm 21×250 or

### C14, 5 μm 21×150 mm

Column temperature: Room Temperature
Flow rate: 20.0 mL/min

### Mobile Phases: A=Water+0.05% TFA

B=ACN+0.05% TFA,
   Injection volume: (100-1500 μL)
   Detector wavelength: 214 nm

Crude compounds were dissolved in 1:1 water:acetic acid at about 50 mg/mL. A 4 minute analytical scale test run was carried out using a 2.1×50 mm C18 column followed by a 15 or 20 minute preparative scale run using 100 μL injection with the gradient based on the % B retention of the analytical scale test run. Exact gradients were sample dependent. Samples with close running impurities were checked with a 21×250 mm C18 column and/or a 21×150 mm C14 column for best separation. Fractions containing desired product were identified by mass spectrometric analysis.

### Analytic HPLC Conditions

### Method A

Column: Agilent Zorbax Bonus-RP C18, 150×4.60 nm, 3.5 micron
Column temperature: 40° C.
Flow rate: 1.5 mL/min
Injection volume: 5 μL
Sample preparation: Dissolve in 1:1 ACN:1 M HCl

### Mobile Phases: A=Water: TFA (99.95:0.05)

B=ACN:TFA (99.95:0.05)
   Detector wavelength: 254 nm and 214 nm
   Gradient: 26 min total (time (min)/% B): 0/5, 18/90, 22/90, 22.5/90, 26/5

### Method B

### Column: Agilent Poroshell 120 Bonus-RP, 4.6×150 mm, 2.7 μm

Column temperature: 30° C.
Flow rate: 1.5 mL/min
Injection volume: 10 μL

### Mobile Phases: A=ACN:Water:TFA (2:98:0.1)

B=ACN:Water:TFA (90:10:0.1)
   Sample preparation: Dissolve in Mobile phase B
   Detector wavelength: 254 nm and 214 nm
   Gradient: 60 min total (time (min)/% B): 0/0, 50/100, 55/100, 55.1/0, 60/0

### Method C

### Column: Agilent Poroshell 120 Bonus-RP, 4.6×150 mm, 2.7 μm

Column temperature: 30° C.
Flow rate: 1.5 mL/min
Injection volume: 10 μL

### Mobile Phases: A=ACN:Water:TFA (2:98:0.1)

B=ACN:Water:TFA (90:10:0.1)
   preparation: Dissolve in Mobile phase B (0.15 mL) then dilute with Mobile phase A (0.85 mL)
   Detector wavelength: 245 nm
   Gradient: 46 min total (time (min)/% B): 0/0, 25/50, 35/100, 40/100, 40.1/0, 46/0

### Example 1: 5-ethyl-2-fluoro-4-(3-(5-(1-methylpiperidin-4-yl)-4,5,6,7-tetrahydro-1H-imidazo[4,5-c]pyridin-2-yl)-1H-indazol-6-yl)phenol, 2HCl Crystalline Form

To Reactor A were added 5-ethyl-2-fluoro-4-(3-(4,5,6,7-tetrahydro-1H-imidazo[4,5-c]pyridin-2-yl)-1H-indazol-6-yl)phenol crystalline hydrate (766 g, 1.55 mol, preparation of the crystalline hydrate shown in U.S. application Ser. No. 15/341,226 filed on Nov. 2, 2016, published as US 2017/0121327), dimethyl sulfoxide (1.38 L), and 200 proof ethanol (3.29 L). With agitation, the slurry was heated to 55° C. and filtered into a clean 20 L Reactor B to remove any undissolved solids. Reactor B was cooled to 25° C. 200 proof ethanol (1.15 L) was added to Reactor A and transferred through filter to Reactor B. The solution in Reactor B was cooled to 25° C. 200 proof ethanol (11.5 L) and 6N hydrochloric acid aqueous solution (0.54 L, 3.27 mol) were added to Reactor A and the mixture was agitated for 5 min at 25° C. The solution in Reactor A was transferred through filter to Reactor B over 4 hrs at 25° C. to produce a suspension. 200 proof ethanol (1.15 L) was added to Reactor A and transferred through filter to Reactor B as a rinse. The suspension in reactor B was agitated for 12 hrs at 25° C., filtered, and washed with 200 proof ethanol (2.68 L). The solids were dried under vacuum at 50° C. overnight to provide 5-ethyl-2-fluoro-4-(3-(5-(1-methylpiperidin-4-yl)-4,5,6,7-tetrahydro-1H-imidazo[4,5-c]pyridin-2-yl)-1H-indazol-6-yl)phenol, 2HCl (809 g, 95% yield) as a white solid. This solid (805 g, 1.47 mol) was added to Reactor B. 200 proof ethanol (15.3 L) and water (0.8 L) were added to Reactor A, agitated for 5 min, and transferred through filter to Reactor B. The suspension in Reactor B was stirred for one day at 40° C. The suspension was cooled to 25° C. over 1 hr, agitated for 2 hrs, then filtered and washed with a mixture of 200 proof ethanol (2.67 L) and water (0.14 L). The solids were dried under vacuum at 50° C. overnight to provide 5-ethyl-2-fluoro-4-(3-(5-(1-methylpiperidin-4-yl)-4,5,6,7-tetrahydro-1H-imidazo[4,5-c]pyridin-2-yl)-1H-indazol-6-yl)phenol, 2HCl crystalline form (771 g, 95% yield) as a white solid.

### Examples 2-8: Properties of the Solid Form

A sample of the crystalline form of the dihydrochloride salt of 5-ethyl-2-fluoro-4-(3-(5-(1-methylpiperidin-4-yl)-4,5,6,7-tetrahydro-1H-imidazo[4,5-c]pyridin-2-yl)-1H-indazol-6-yl)phenol of Example 1 was analyzed by powder X-ray diffraction (PXRD), differential scanning calorimetry (DSC), thermogravimetric analysis (TGA), dynamic moisture sorption (DMS), single crystal X-Ray diffraction, and chlorine ion determination. The stability and solubility of the form were also determined.

### Example 2: Powder X-Ray Diffraction

The powder X-ray diffraction patterns of FIG. 1 was obtained with a Bruker D8-Advance X-ray diffractometer using Cu-Kα radiation (λ=1.54051 Å) with output voltage of 45 kV and current of 40 mA. The instrument was operated in Bragg-Brentano geometry with incident, divergence, and scattering slits set to maximize the intensity at the sample. For measurement, a small amount of powder (5-25 mg) was gently pressed onto a sample holder to form a smooth surface and subjected to X-ray exposure. The samples were scanned in 2θ-2θ mode from 2° to 35° in 2θ with a step size of 0.02° and a scan speed of 0.30° seconds per step. The data acquisition was controlled by Bruker DiffracSuite measurement software and analyzed by Jade software (version 7.7). The instrument was calibrated with a corundum standard, within ±0.02° two-theta angle. Observed PXRD 2θ peak positions and d-spacings are shown in Table 1.

TABLE 1
PXRD Data for the Crystalline Form

| 2θ | d(Å) | Area |
|---|---|---|
| 5.99 | 6.06 | 3.4 |
| 7.74 | 4.69 | 0.2 |
| 8.48 | 4.29 | 0.6 |
| 11.98 | 3.03 | 4.5 |
| 12.47 | 2.92 | 2.4 |
| 13.11 | 2.77 | 0.3 |
| 15.12 | 2.41 | 0.8 |
| 15.53 | 2.34 | 1 |
| 17.67 | 2.06 | 6.2 |
| 18.02 | 2.02 | 14.4 |
| 19.45 | 1.87 | 0.5 |
| 19.77 | 1.84 | 0.5 |
| 20.64 | 1.77 | 0.7 |
| 21.22 | 1.72 | 2.6 |
| 21.48 | 1.70 | 1.5 |
| 22.17 | 1.65 | 3.7 |
| 23.66 | 1.54 | 1.2 |
| 24.99 | 1.46 | 2.1 |
| 26.84 | 1.36 | 1 |
| 27.29 | 1.34 | 1.6 |
| 28.13 | 1.30 | 1.3 |

### Example 3: Thermal Analysis

Differential scanning calorimetry (DSC) was performed using a TA Instruments Model Q-100 module with a Thermal Analyst controller. Data were collected and analyzed using TA Instruments Thermal Analysis software. A sample of the crystalline form was accurately weighed into a covered aluminum pan. After a 5 minutes isothermal equilibration period at 5° C., the sample was heated using a linear heating ramp of 10° C./min from 0° C. to 335° C. A representative DSC thermogram of the crystalline form is shown in FIG. 2. The crystalline form was found to have a melting temperature with an onset at 312.6° C., and a peak endotherm at 325.7° C. The thermogram shows overlapping of melting and decomposition.

Thermogravimetric analysis (TGA) measurements were performed using a TA Instruments Model Q-50 module equipped with high resolution capability. Data were collected using TA Instruments Thermal Analyst controller and analyzed using TA Instruments Universal Analysis software. A weighed sample was placed onto a platinum pan and scanned with a heating rate of 10° C. from ambient temperature to 360° C. The balance and furnace chambers were purged with nitrogen flow during use. A representative TGA trace of the crystalline form is shown in FIG. 3. The crystalline form decomposes at an onset temperature of about 285° C.

### Example 4: Dynamic Moisture Sorption Assessment

Dynamic moisture sorption (DMS) measurement was performed using a VTI atmospheric microbalance, SGA-100 system (VTI Corp., Hialeah, Fla. 33016). A weighed sample was used and the humidity was at the lowest possible value (close to 0% RH) at the start of the analysis. The DMS analysis consisted of an initial drying step (˜0% RH) for 16 hours, followed by two cycles of sorption and desorption with a scan rate of 5% RH/step over the humidity range of 5% RH to 90% RH. The DMS run was performed isothermally at 25° C. A representative DMS trace for the crystalline form is shown in FIG. 4. The crystalline form exhibits a total moisture uptake of about 0.8% when exposed to a range of relative humidity between about 5% and about 90% at room temperature. There was no change in form after two cycles of moisture sorption and desorption.

### Example 5: Single Crystal X-Ray Diffraction

A crystal of the crystalline form having dimensions of 0.11×0.05×0.1 mm was mounted on a nylon loop. Data were collected on a Rigaku Atlas CCD diffractometer equipped with an Oxford Cryosystem Cobra cooling device. The data were collected using Cu Kα radiation and the crystal structure was solved and refined using the Bruker AXS SHELXTL software. Hydrogen atoms attached to carbon were placed geometrically and allowed to refine with a riding isotropic displacement parameter. Hydrogen atoms attached to the heteroatoms were located in a difference Fourier map and were allowed to refine freely with an isotropic displacement parameter. Unit cell parameters, along with crystal system and space group details, are provided in Table 2.

TABLE 2
Single Crystal X-Ray Diffraction Analysis Data

| | |
|---|---|
| Temperature of Data Collection | 293(2) K |
| Wavelength used for Data Collection | 1.54184 Å |
| Crystal system | Monoclinic |
| Space group | P2₁/c |
| Unit cell dimensions | a = 15.2725(8) Å |
| | b = 7.6112(6) Å |
| | c = 23.6525(13) Å |
| | α = 90° |
| | β = 105.105(6)° |
| | γ = 90° |
| Unit cell volume | 2654.4(3) Å³ |
| Final R indices [F² > 2σ(F²)] | R₁ = 0.0520, wR₂ = 0.1240 |

### Example 6: Stability Study

Samples of the crystalline form were stored in glass vials with screw caps at 40° C. and 75% relative humidity (RH). At specific intervals, the contents of a representative sample were removed and analyzed by HPLC for assay and chemical purity as shown in Table 3. There was no significant change in purity or assay of the dihydrochloride salt form after 6 months at 40° C. and 75% RH, indicating that this salt form is stable.

TABLE 3
Crystalline Dihydrochloride Salt Form Stability Results

| | | T = 1 | T = 2 | T = 4 | T = 3 | T = 6 |
|---|---|---|---|---|---|---|
| | T = 0 | Week | Weeks | Weeks | Months | Months |
| HPLC purity % a/a | 98.7 | 98.7 | 98.7 | 98.7 | 98.7 | 98.5 |
| Assay % w/w | 85.0 | 86.8 | 85.1 | 86.9 | 84.5 | 85.1 |

### Example 7: Chloride Ion Determination

The chloride content was determined using ion chromatography and conductivity detection. A sample of the crystalline form was dissolved in 10% acetonitrile and assayed against a chloride standard solution prepared at 25 microg/mL, using a Dionex IonPac AS20 column installed on a Dionex ICS-2100 ion chromatography system. The chloride content was found to be 12.6% w/w, which confirms that the crystalline form is that of the dihydrochloride salt of compound 1.

### Example 8: Solubility of the Crystalline Form in Water and Simulated Lung Fluid

The solubility of the crystalline form was assessed in water and Simulated Lung Fluid (SLF). The crystalline form was dispersed in liquid media in multiple vials to form slurries, with each vial containing about 7.5 mg of the crystalline form and 0.5 mL of the liquid media. The slurries were agitated in a thermo-shaker at 25° C. and samples were collected from different vials at approximately 5, 15, 30, 60 minutes, and 24 hours. At each of the timepoints, the slurries were centrifuged for 10 minutes. The supernatant was collected and centrifuged for another 10 minutes. Solubility at each timepoint is determined by analyzing the compound concentration in the supernatant using HPLC. SLF composition: 0.4% NaCl, 0.02% DPPC in 50 mM phosphate buffer pH 6.9

The crystalline form's solubility in SLF increased over the first 30 minutes to 0.21 mg/mL and then decreased to 0.07 mg/mL at 24 hours. The starting pH of the SLF was 6.9; it remained nearly unchanged, with pH 6.8 recorded during the first 60 minutes. At 24 hours, the pH was lowered to 5.1.

The crystalline form's solubility in water increased over the first 60 minutes to 8.9 mg/mL and then decreased to 3.2 mg/mL at 24 hours. The pH of water was lowered to 4.0 at all timepoints during the study.

While the present disclosure provides reference to specific aspects or embodiments, it will be understood by those of ordinary skilled in the art that various changes can be made or equivalents can be substituted without departing from the true spirit and scope of the disclosure. Additionally, to the extent permitted by applicable patent statutes and regulations, all publications, patents and patent applications cited herein are hereby incorporated by reference in their entirety to the same extent as if each document had been individually incorporated by reference herein.

1. A crystalline form of the dihydrochloride salt of 5-ethyl-2-fluoro-4-(3-(5-(1-methylpiperidin-4-yl)-4,5,6,7-tetrahydro-1H-imidazo[4,5-c]pyridin-2-yl)-1H-indazol-6-yl)phenol characterized by a powder X-ray diffraction pattern comprising diffraction peaks at 2θ values of 5.99±0.2, 11.98±0.2, 17.67±0.2, and 18.02±0.2.

2. The crystalline form of claim 1, wherein the powder X-ray diffraction pattern is further characterized by having one additional diffraction peak at a 2θ value of 22.17±0.2.

3. The crystalline form of claim 2, wherein the powder X-ray diffraction pattern is further characterized by having two additional diffraction peaks at 2θ values of 12.47±0.2, and 21.22±0.2.

4. The crystalline form of claim 3, wherein the powder X-ray diffraction pattern is further characterized by having two additional diffraction peaks at 2θ values of 7.74±0.2, and 8.48±0.2.

5. The crystalline form of claim 1, wherein the powder X-ray diffraction pattern is further characterized by having two or more additional diffraction peaks at 2θ values selected from 7.74±0.2, 8.48±0.2, 12.47±0.2, 21.22±0.2, and 22.17±0.2.

6. The crystalline form of claim 4, wherein the powder X-ray diffraction pattern is further characterized by having two or more additional diffraction peaks at 2θ values selected from 13.11±0.2, 15.12±0.2, 15.53±0.2, 19.45±0.2, 19.77±0.2, 20.64±0.2, 21.48±0.2, 23.66±0.2, 24.99±0.2, 26.84±0.2, 27.29±0.2, and 28.13±0.2.

7. The crystalline form of claim 1, wherein the crystalline form is characterized by a powder X-ray diffraction pattern in which the peak positions are substantially in accordance with the peak positions of the pattern shown in FIG. 1.

8. The crystalline form of claim 1, wherein the crystalline form is characterized by a differential scanning calorimetry trace recorded at a heating rate of 10° C. per minute which shows a maximum in endothermic heat flow at a temperature of 325.7±2° C.

9. The crystalline form of claim 1, wherein the crystalline form is characterized by a differential scanning calorimetry trace recorded at a heating rate of 10° C. per minute which shows a melting endotherm with an onset of 312.6±2° C.

10. The crystalline form of claim 1, wherein the crystalline form is characterized by a differential scanning calorimetry trace substantially in accordance with that shown in FIG. 2.

11. A pharmaceutical composition comprising the crystalline form of claim 1 and a pharmaceutically-acceptable carrier.

12. The pharmaceutical composition of claim 11 which is a dry powder suitable for inhalation.

13. A method of preparing the crystalline form of claim 1 comprising:
(a) heating at 55° C.±10° C. 5-ethyl-2-fluoro-4-(3-(4,5,6,7-tetrahydro-1H-imidazo[4,5-c]pyridin-2-yl)-1H-indazol-6-yl)phenol or a hydrate or solvate thereof, in DMSO and ethanol, wherein the ratio of DMSO:ethanol is 1:2 to 3, to give a mixture,
(b) cooling off the mixture obtained in step (a) to about 25° C. and adding 2 to 2.5 equivalents of hydrochloric acid in ethanol at 25° C.±10° C. to produce a suspension,
(c) filtering the suspension of step (b) to give a solid,
(d) adding ethanol and water at a ratio of ethanol:water of 15 to 25:1 to the solid of step (c) and stirring the mixture obtained at 25° C.±10° C. for 12-36 hours to give a suspension, and
(e) isolating the crystalline form from the suspension of step (d).

14. The method of claim 13 wherein the method involves drying the solid of step (c) at 50° C.±10° C. before performing step (d).

15.-40. (canceled)

41. A method of treating a respiratory disease in a mammal, the method comprising administering to the mammal the crystalline form of claim 1 and a pharmaceutically-acceptable carrier.

42. The method of claim 41, wherein the respiratory disease is selected from the group consisting of asthma, chronic obstructive pulmonary disease, cystic fibrosis, pneumonitis, idiopathic pulmonary fibrosis, acute lung injury, acute respiratory distress syndrome, bronchitis, emphysema, sarcoidosis, an eosinophilic disease, a helminthic infection, pulmonary arterial hypertension, lymphangioleiomyomatosis, bronchiectasis, an infiltrative pulmonary disease, drug-induced pneumonitis, fungal induced pneumonitis, allergic bronchopulmonary aspergillosis, hypersensitivity pneumonitis, eosinophilic granulomatosis with polyangiitis, idiopathic acute eosinophilic pneumonia, idiopathic chronic eosinophilic pneumonia, hypereosinophilic syndrome, Löffler syndrome, bronchiolitis obliterans organizing pneumonia, lung graft-versus-host disease, and immune-checkpoint-inhibitor induced pneumonitis.

43. The method of claim 41, wherein the respiratory disease is asthma.

44. The method of claim 43, wherein the asthma is moderate to severe asthma, Th2 high asthma, or Th2 low asthma.

45.-46. (canceled)

47. The method of claim 41, wherein the crystalline form is administered by inhalation.

48. A method of preventing or delaying lung transplant rejection in a mammal, the method comprising administering to the mammal the crystalline form of claim 1, and a pharmaceutically-acceptable carrier.

49. The method of claim 48, wherein the lung transplant rejection is selected from the group consisting of primary graft dysfunction, organizing pneumonia, acute rejection, lymphocytic bronchiolitis, and chronic lung allograft dysfunction.

50. The method of claim 48, wherein the lung transplant rejection is acute lung transplant rejection, chronic lung allograft dysfunction, bronchiolitis obliterans, restrictive chronic lung allograft dysfunction, or neutrophilic allograft dysfunction.

51.-52. (canceled)

53. The method of claim 48, wherein the crystalline form is administered by inhalation.
